(19) United States
(12) Patent Application Publication
(10) Pub. No.: US 2023/0199361 A1
(43) Pub. Date: Jun. 22, 2023

(54) Headphone Earcup Cover Device

(71) Applicants: Faith Uti, Ramsey, NJ (US); Abby Tom, Ramsey, NJ (US)

(72) Inventors: Faith Uti, Ramsey, NJ (US); Abby Tom, Ramsey, NJ (US)

(21) Appl. No.: 18/062,162

(22) Filed: Dec. 6, 2022

Related U.S. Application Data
(60) Provisional application No. 63/291,610, filed on Dec. 20, 2021.

(51) Int. Cl. H04R 1/10

(52) U.S. Cl. CPC H04R 1/1008

(57) ABSTRACT

The present invention relates to a headphone earcup cover device that increases the comfort and functionality of the earcups of a pair of over-ear headphones. The device is primarily comprised of a body with a first opening and a second opening. The device is comprised of a fabric material that in different embodiments is comprised of a warming fabric material and/or a moisture-wicking, water-resistant material. The device can be secured around an earcup of a headphone via an elastic band, drawstring, or other fastener. The device may also be comprised of a padding to increase comfort.

### CROSS-REFERENCE TO RELATED APPLICATION

The present application claims priority to, and the benefit of, U.S. Provisional Application No. 63/291,610 which was filed on Dec. 20, 2021, and is incorporated herein by reference in its entirety.

### FIELD OF THE INVENTION

The present invention relates generally to the field of headphone covers. More specifically, the present invention relates to a headphone earcup cover device primarily comprised of a body with a first opening and a second opening. The device is comprised of a fabric material that, in different embodiments, is comprised of a warming fabric material and/or a moisture-wicking, water-resistant material. The device can be secured around an earcup of a headphone via an elastic band, drawstring, or other fastener. The device may also be comprised of a padding to increase comfort. Accordingly, the present disclosure makes specific reference thereto. Nonetheless, it is to be appreciated that aspects of the present invention are also equally applicable to other like applications, devices, and methods of manufacture.

### BACKGROUND

Standard over-the-ear headphones typically feature leather earcup cushions. However, said cushions can become uncomfortable if worn for a long period. Further, the leather cushion can become dry, cracked, and damaged after prolonged use. Sensitive skin can be affected and develop rashes and discomfort due to direct contact with the leather cushion.

In addition, leather earcup cushions do not typically have a warming effect on the ears of the wearer. As a result, a user's ears can become cold while wearing headphones with leather earcups in a cold environment. This is undesirable as the user may then opt to not wear the headphones.

Similarly, leather earcup cushions are not typically waterproof or water-resistant. As a result, leather earcup cushions can be easily damaged by sweat if a user wears headphones when working out. In addition, sweat can collect on the leather earcup creating an uncomfortable sensory experience for the wearer.

Therefore, there exists a long-felt need in the art for a device that improves the usability of a headphone earcup. There also exists a long-felt need in the art for a headphone earcup cover device that improves the comfort of a headphone earcup. There also exists a long-felt need in the art for a headphone earcup cover device that warms the ears of a user while in use. In addition, there exists a long-felt need in the art for a headphone earcup cover device that protects the earcup of a headphone from sweat and/or water.

The subject matter disclosed and claimed herein, in one embodiment thereof, comprises a headphone earcup cover device. The device is primarily comprised of a body with a first opening and a second opening. The device is comprised of a fabric material that in different embodiments are comprised of a warming fabric material and/or a moisture-wicking, water-resistant material. The device can be secured around an earcup of a headphone via an elastic band, drawstring, or other fastener. In one embodiment, the device is comprised of a warming exterior material and a moisture-wicking interior material such that the device is reversible. The device may also be comprised of a padding to increase comfort.

In this manner, the headphone earcup cover device of the present invention accomplishes all of the foregoing objectives and provides a device that improves the usability of a headphone earcup. During use, the device improves the comfort of a headphone earcup. In one embodiment, the device can also warm the ear of a user while in use. Further, the device protects the earcup of a headphone from sweat and/or water.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a headphone earcup cover device. The device is primarily comprised of a body with a first opening and a second opening

Accordingly, the headphone earcup cover device of the present invention is particularly advantageous as it provides a device that improves the usability of a headphone earcup. During use, the device improves the comfort of a headphone earcup. In various embodiments, the device can warm the ear of a user while in use and may also protect the earcup of a headphone from sweat and/or water. In this manner, the headphone earcup cover device overcomes the limitations of existing headphone earcup covers known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a headphone earcup cover device of the present invention in accordance with the disclosed architecture; and

FIG. 2 illustrates a perspective view of one potential embodiment of a headphone earcup cover device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that improves the usability of a headphone earcup. There also exists a long-felt need in the art for a headphone earcup cover device that improves the comfort of a headphone earcup. There also exists a long-felt need in the art for a headphone earcup cover device that warms the ears of a user while in use. In addition, there exists a long-felt need in the art for a headphone earcup cover device that protects the earcup of a headphone from sweat and/or water.

The present invention, in one exemplary embodiment, is comprised of a headphone earcup cover device.

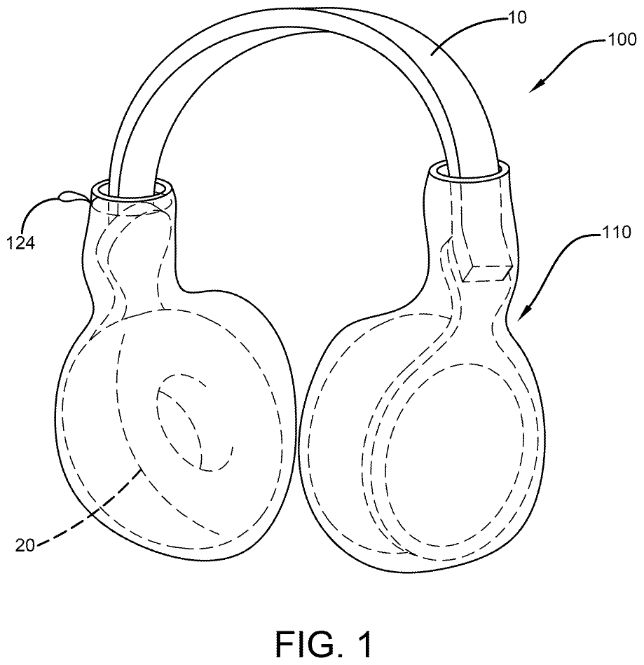

Referring initially to the drawings, FIG. 1 illustrates a perspective view of one potential embodiment of a headphone earcup cover device 100 of the present invention in accordance with the disclosed architecture. The device 100 is primarily comprised of a body 110 with a first opening 120 and a second opening 130. The body 110 is preferably made from a fabric material. Said material includes but is not limited to vinyl, canvas, cashmere, chenille, chiffon, cotton, damask, jersey, lace, linen, wool, modal, polyester, satin, silk, spandex, suede, tweed, twill, velvet, acrylic, modacrylic, nylon, polypropylene, polyurethane, polyvinyl chloride, polyethylene, vinylidene, benzoate, aramid, rayon, acetate, triacetate, etc. However, the fabric material may differ in various embodiments.

In one embodiment, the fabric material of the body 110 is comprised of a warming material such as wool, fleece, sherpa, etc. However, the body 110 may be comprised of any warming fabric known in the art. In this manner, the body 110 keeps the ears of the user warm while the device 100 is placed over the earcups 20 of a pair of headphones 10.

In a differing embodiment, the fabric material of the body 110 is comprised of a moisture-wicking material. This embodiment of the body 110 prevents sweat from collecting on the body 110 as a user is working out while wearing headphones 10 with a device 100 attached over each earcup 20. Said material may also be waterproof or water-resistant to protect the earcups 20 against sweat.

The body 110 may also be comprised of at least one type of padding 170. The padding 170 increases the comfort of the device 100 while worn. In this manner, the device 100 provides increased comfort over the existing earcup 20 material. The padding 170 may be comprised of a memory foam or other material known in the art.

In another embodiment, the body 110 may be comprised of a first material for the exterior material 112 of the body 110 and a second material for the interior material 114 of the body 110. In this manner, the body 110 is reversible. In one exemplary embodiment, the exterior material 112 is a warming material and the interior material 114 is a moisture-wicking material. As a result, the body 110 is reversible such that the exterior material 112 or interior material 114 can contact the ears of the wearer during use, based on the usage application (i.e., staying warm or working out).

In any embodiment of the device 100, the body 110 (including the exterior material 112 and interior material 114) may be comprised of at least one indicia 140. The indicia 140 may include but is not limited to patterns, logos, emblems, images, symbols, designs, letters, numbers, words, characters, animals, advertisements, brands, etc. The indicia 140 of the exterior material 112 may differ from the interior material 114 to allow the device 100 to have two different appearances. In this embodiment, the exterior material 112 and interior material 114 may be made from the same material (i.e., to perform the same function) or different material (i.e., to perform different functions).

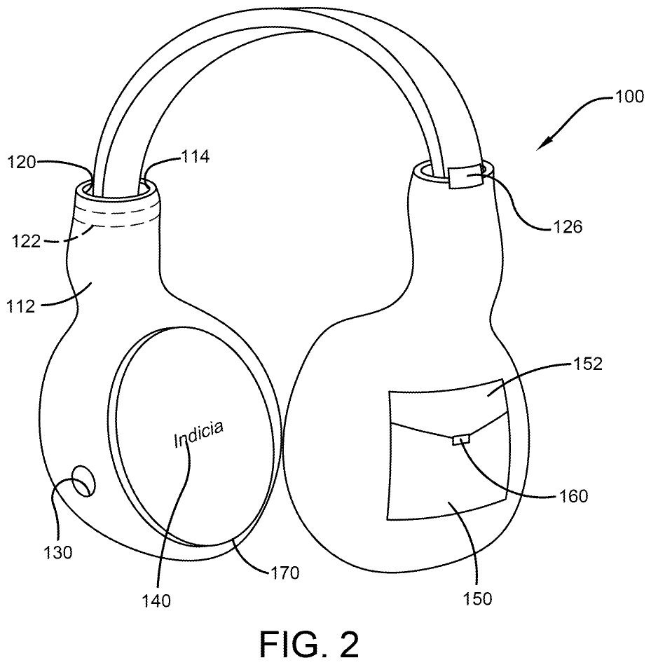

The body 110 may also be comprised of at least one pocket 150, as seen in FIG. 2. The pocket 150 allows a user to store various items such as, but not limited to, keys, a charging cord, etc. The pocket 150 may be comprised of at least one pocket fastener 160. In one embodiment, the pocket 150 is comprised of at least one flap 152 that can be secured to the pocket 150 via the fastener 160 to secure the contents of the pocket 150. The fastener 160 may be any fastener known in the art such as, but not limited to, hook and loop, magnet, snap button, zipper, etc.

The body 110 is also comprised of a first opening 120. The first opening 120 allows the body 110 to be placed over an earcup 20 of a pair of headphones 10 such that the body 110 encapsulates the earcup 20. This also protects the earcup 20 against damage. In one embodiment, the opening 120 is comprised of at least one internal elastic band 122. The band 122 tightens the opening 120 around the earcup 20 such that the device 100 cannot become dislodged.

In another embodiment, the opening 120 is comprised of at least one drawstring 124. The drawstring 124 may be of any type or style known in the art. The drawstring 124 tightens the opening 120 around the earcup 20 such that the device 100 cannot become dislodged.

However, it should be appreciated that the opening 120 may be comprised of at least one fastener 126 of any type known in the art located on the exterior material 112 and/or interior material 114. The fastener 126 tightens the opening 120 around the earcup 20 such that the device 100 cannot become dislodged. The fastener 126 may include, but is not limited to, hook and loop, magnet, snap button, ties, etc. Alternatively, the fastener 126 may secure the device 100 to the headphone 10.

The body 110 may also be comprised of at least one second opening 130. The second opening 130 may be located anywhere on the body 110. The second opening 130 allows the charging port and/or controls of the earcup 20 to still be accessed while the device 100 is placed over the earcup 20.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “headphone earcup cover device” and “device” are interchangeable and refer to the headphone earcup cover device 100 of the present invention.

Notwithstanding the foregoing, the headphone earcup cover device 100 of the present invention and its various components can be of any suitable size and configuration as is known in the art without affecting the overall concept of the invention, provided that they accomplish the above-stated objectives. One of ordinary skill in the art will appreciate that the size, configuration and material of the headphone earcup cover device 100 as shown in the FIGS. are for illustrative purposes only, and that many other sizes and shapes of the headphone earcup cover device 100 are well within the scope of the present disclosure. Although the dimensions of the headphone earcup cover device 100 are important design parameters for user convenience, the headphone earcup cover device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A headphone earcup cover device comprising:
a body;
a first opening; and
a fastener.

2. The headphone earcup cover device of claim 1, wherein the body is comprised of a fabric material.

3. The headphone earcup cover device of claim 2, wherein the fabric material is comprised of a wool, a fleece, or a sherpa material.

4. The headphone earcup cover device of claim 1, wherein the fastener tightens the first opening.

5. The headphone earcup cover device of claim 4, wherein the fastener is comprised of an elastic band.

6. A headphone earcup cover device comprising:
a body;
a first opening;
a second opening; and
a fastener.

7. The headphone earcup cover device of claim 6, wherein the fastener is comprised of a drawstring.

8. The headphone earcup cover device of claim 6 comprised of a pocket.

9. The headphone earcup cover device of claim 6, wherein the body is comprised of a reversible material.

10. A headphone earcup cover device comprising:
a body;
a padding;
a first opening;
a pocket;
a second opening; and
a first fastener.

11. The headphone earcup cover device of claim 10, wherein the body is comprised of an exterior material and an interior material.

12. The headphone earcup cover device of claim 11, wherein the exterior material is comprised of a warming fabric.

13. The headphone earcup cover device of claim 11, wherein the interior material is comprised of a moisture-wicking fabric.

14. The headphone earcup cover device of claim 13, wherein the moisture-wicking fabric is comprised of a waterproof material.

15. The headphone earcup cover device of claim 10, wherein the padding is comprised of a memory foam padding.

16. The headphone earcup cover device of claim 10, wherein the pocket is comprised of a flap.

17. The headphone earcup cover device of claim 16, wherein flap is comprised of a second fastener that secures the flap to the pocket.

18. The headphone earcup cover device of claim 17, wherein the second fastener is comprised of a hook and loop fastener, a magnet fastener, a snap button fastener, or a zipper fastener.

19. The headphone earcup cover device of claim 10, wherein the first fastener is comprised of a hook and loop fastener, a magnet fastener, a snap button faster, or a tie fastener.

20. The headphone earcup cover device of claim 11, wherein the first fastener is positioned on the interior material.
